(19) United States
(12) Patent Application Publication
(10) Pub. No.: US 2010/0298649 A1
(43) Pub. Date: Nov. 25, 2010

(54) SYSTEM AND METHODS FOR ASSESSMENT OF THE AGING BRAIN AND ITS BRAIN DISEASE INDUCED BRAIN DYSFUNCTIONS BY SPEECH ANALYSIS

(76) Inventors: Siegbert Warkentin, Limhamn (SE); Catarina Erikson, Limhamm (SE)

Correspondence Address: KNOBBE MARTENS OLSON & BEAR LLP, 2040 MAIN STREET, FOURTEENTH FLOOR, IRVINE, CA, 92614, US

(21) Appl. No.: 12/741,023

(22) PCT Filed: Nov. 3, 2008
(86) PCT No.: PCT/EP2008/064891

§ 371 Date: Jul. 23, 2010

Related U.S. Application Data
(60) Provisional application No. 60/984,934, filed on Nov. 2, 2007.

(30) Foreign Application Priority Data
Nov. 2, 2007 (DE) SE0702425-0

(51) Int. Cl. A61B 5/00; G01N 33/00

(52) U.S. Cl. 600/300; 436/76; 436/98

(57) ABSTRACT

A system and method for assessment of a brain status of a subject are disclosed. The brain status comprises a brain disease induced brain dysfunction. An occurrence and/or stage of the brain disease induced brain dysfunction in the subject is determined. The system comprises an apparatus that is adapted to determine the occurrence and/or stage of the brain disease induced brain dysfunction in the subject from random speech of the subject. The apparatus (200) comprises units that are operatively connected to each other, which comprises a unit (205) for registering the speech of the subject over a period of time; a unit (206) devised for analyzing the registered speech and configured to determine a pause component of the speech; and a unit that is adapted to determine the occurrence and/or stage of the brain disease induced brain dysfunction from the pause component, wherein said pause component is an accumulated pause time of a total time of said speech correlated to said occurrence and/or stage of said brain dysfunction.

### FIELD OF THE INVENTION

This invention pertains in general to the field of systems and methods for assessment of a brain disease induced brain dysfunction which is not of developmental origin of the central nervous system (CNS), but reflects the aging and disease processes of the CNS in the elderly. More particularly the invention relates to such systems and methods for determining or diagnosing if the person suffers from a brain disease induced brain dysfunction or is in risk thereof by analyzing speech of the person.

### BACKGROUND OF THE INVENTION

The brain may be damaged in many various ways by the aging CNS, and CNS-changes may precede clinical evidence of such changes for many decades. This can be seen for example in mild cognitive impairment (MCI), small or large vessel diseases, damage of the blood brain barrier function, atherosclerosis, etc. where clinical symptoms of ongoing brain damaging processes may not be evident until a certain point is reached in the development of such processes.

Diseases, in which such processes are accelerated and clinically evident comprise for instance dementia, Alzheimer's disease (AD); or Multiple sclerosis (MS), but includes also many other CNS-diseases.

A large number of persons are affected by such diseases. Dementia and dementia-associated diseases are actually ranked as the fourth common cause of death in industrialized civilizations of the globe, after cardiac diseases, cancer, and stroke. In Sweden alone, having a population of only nine million, about 150000-200,000 persons are suffering from dementia. About 7 percent of the elderly and 20-30 percent of the 85 year old persons suffer from dementia. As the percentage of elderly of the total population will increase due to longer expected life, the absolute number of patients will even increase with time. Therefore, there is a need to identify persons at risk of developing dementia or having a certain degree of dementia as early as possible in order to be able to provide suitable treatment.

Computerized tomography (CT-scan) and MRI (magnetic resonance imaging) are today widely used in the clinical assessment of brain diseases and also for the assessment of white matter abnormalities. For the assessment of brain functional disturbances and cerebrovascular disorder SPECT (Single Photon Emission Tomography) is used in routine clinical practice, while other techniques like PET (Positron Emission Tomography), fMRI (functional MRI), etc., are mainly used for research purposes to assess cerebral blood flow, brain metabolic processes, and neurotransmitter function.

A problem with structural and functional brain imaging methods is that they do not provide information about the subject's cognitive difficulties.

The behavioural consequences of brain diseases may be tested by cognitive testing. Cognitive testing of subjects running a risk for developing dementia, for example subjects with mild cognitive impairment, MCI, is usually performed by psychologists in specialist settings and is time consuming. Primary care physicians, nurses, and occupational therapists have little time to perform cognitive assessment, and widely use standard instruments, like the MiniMental State Examination (MMSE), which is limited by educational and cultural factors, as are all tests using cognitive content questions.

Automated Systems implementing such cognitive testing have been disclosed, e.g. in US2006/0194176A1 or EP1205146.

In US2006/0194176A1 a dementia testing apparatus e.g. for senile dementia, is disclosed, which has a test chart that comprises tale including test sentences containing colored words and questions for determining whether words are colored with a color expressed by a colored word. In more detail, an answer obtaining section of the apparatus obtains answers from a patient that are made within predetermined answer time limits to a first and a second examination chart. The first examination chart has inspection sentences where a character group constituting a story including color words each representing color is tinted with plural colors such that individual color word has characters of the same color, requires a determination as to whether the color of characters constituting the color word is the same color as color represented by the color word. The second examination chart has a combination of questions concerning contents of the inspection sentences and answers which are prepared for each question and one of which is to be selected. In a dementia degree inspection section of the apparatus, a dementia degree of the subject based on the answers obtained by the answer obtaining section is determined.

In EP1205146 a patient answer based dementia test system for testing the degree of dementia of a subject is disclosed. The dementia test system which is effective for preventing and finding, at an early stage, an initial sign (initial dementia) of senile dementia. A dementia test apparatus comprising an answer obtaining section for obtaining an answer of a subject to both a dementia degree test chart which requires the subject to exercise a plurality of judgments at the same time and obtain an answer in such a form that correction of judgment is objectively determined, and a dementia factor degree test chart comprising a combination of multiple questions concerning sensibility and a multiplicity of answers alternatively selected from questions prepared for each of the former questions, and a dementia degree test section for testing a dementia degree indicative of the current degree of dementia of the subject based on an answer obtained by the answer obtaining section, and for estimating a future dementia degree of the subject.

However, the test systems of the prior art, such as disclosed in US2006/0194176A1 or EP1205146, suffer from the same drawbacks as manually performed cognitive tests, e.g. a dependency of the test on educational, social and cultural factors, including language, of the subject.

Thus, there is a need for a new or improved system and/or method for assessing brain damages caused by brain diseases or a risk for developing such diseases. It is desired that such a system and/or method is providing a reliable diagnosis of brain damage induced brain dysfunctions independent of educational, social and cultural factors, including language, of the subject to be diagnosed.

Hence, an improved system and/or method, e.g. for assessing brain damage caused by diseases of the aging brain or a risk for developing such brain damage, would be advantageous and in particular a system and/or method allowing for increased flexibility, cost-effectiveness, patient comfort and/or independency of educational background and/or cultural factors and language of a subject to be tested, would be advantageous.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing a system, a method, a computer program, and a medical workstation according to the appended patent claims.

Random speech is registered and analyzed. Correlations between an accumulated pause time in relation to the total speech time and brain damage induced brain damages are analyzed for a diagnosis.

According to a first aspect of the invention, a system is provided, wherein the system is devised for assessment of a brain status of a subject, and wherein the brain status comprises a brain disease induced brain damage. The system is adapted to determine an occurrence and/or stage of the brain disease induced brain dysfunction in the subject. The system comprises an apparatus that is adapted to determine the occurrence and/or stage of the brain disease induced brain damage in the subject from speech of the subject. The speech may be random speech of the subject. Alternatively, or in addition, the speech may be based on a naming task, which is arranged and performed independent of the subject's language. The apparatus comprises units that are operatively connected to each other, which comprises a unit for registering the speech of the subject over a period of time; a unit devised for analyzing the registered speech and configured to determine a pause component of the speech; and a unit that is adapted to determine the occurrence and/or stage of the brain disease induced brain damage from the pause component. The pause component is an accumulated pause time obtained during the total time of said speech, which pause component is correlated to said occurrence and/or stage of said brain dysfunction.

According to a second aspect of the invention, a method for assessment of a brain status of a subject is provided, wherein the brain status comprises a brain damage induced by a brain disease. The method comprises analyzing speech of the subject and determining the aforementioned pause component of the speech; and determining an occurrence and/or stage of the brain damage induced by the brain disease in the subject based on the pause component.

According to a third aspect of the invention, a computer program for processing by a computer is provided. The computer program is configured for assessment of a brain status of a subject, wherein the brain status comprises a brain damage induced by a brain disease. The computer program comprises a first code segment for analyzing speech of the subject and determining the accumulated pause duration of the speech as defined herein; and a second code segment for determining an occurrence and/or stage of the brain damage induced by brain disease in the subject based on this pause component.

According to a further aspect of the invention, a medical workstation is provided, wherein the medical workstation is adapted for executing the computer program according to the third aspect of the invention.

Further embodiments of the invention are defined in the dependent claims, wherein features for the second and subsequent aspects of the invention are as for the first aspect mutatis mutandis.

Some embodiments of the invention provide for the following advantages, alone or in any combination, depending on the specific embodiments:
Measures of processing speed (such as for example using simple colors and shapes, or naming other defined stimuli) are non-invasive, i.e. such tests are easily tolerated by subjects without offending them. In fact, patients are unaware whether they performed good or bad on the test. This is in contrast to knowledge questions raised by the MMSE, where patients often become painfully aware of their cognitive problems.
Embodiments of this invention are implemented in an education and culture-free manner, primarily due to the fact that no knowledge questions are asked. The age-effect is minimal, and lies well within the boundaries of the cut-off limit between normal speed and pathological slowing.
The assessment of accumulated pause time by embodiments of this invention is the most sensitive measure of information processing speed (which was hitherto not known and wherein a detailed reasoning and example study proving this fact is described in detail further below).
From the primary health care perspective, doctors, nurses or occupational therapists are not offended by using this innovation.
On the contrary, they are provided with a powerful tool allowing them to rationalize their work and to concentrate on subjects in need of therapeutic care. Based on the assessment results provided by embodiments of the present invention, doctors may easily decide which patients have signs of a decline in processing speed and therefore are at risk for developing a brain disease induced brain dysfunction, or not. This information may therefore direct primary health care resources to those patients who are at high risk for having a brain disorder, and who need further assessment for their diagnosis, while saving financial costs for unnecessary evaluations of patients with negative test results.
A negative test result provided by an embodiment of this invention saves time and worry on behalf of the patient, and is positive information if the patient (or the relative) has e.g. been worried about beginning AD. A negative test result should at the discretion of a doctor, however, be accompanied by a routine clinical evaluation and laboratory screening in order to rule out physical illness.
Based on experience from individual cases, the speed measure has sometimes been the only measure (including blood tests, MMSE etc.), which has been decisive for further assessment of the patient's complaints of possible brain disease. On the basis of a positive test result solely based on the speed measure, patients have finally received an objective confirmation and a clinical diagnosis (leucoaraiosis, subclinical white matter infarcts, etc.).
Some embodiments are cost effective, as e.g. a test session takes a few minutes to perform. This is in practice an essential point as time allocation for each patient in primary health care is short.
Some embodiments are cost effective and convenient to perform as a handheld apparatus may implement self-testing by said subject.
The automatized voice recording and analysis of the test results makes the measure objective and independent of an examiner. It works much like a laboratory test.
Baseline evaluation of test results obtained by some embodiments at a first visit to the doctor may be used as reference values at successive visits. This makes it possible to capture whether progress (cognitive slowing) has occurred over time. If this is the case and the subject shows a cognitive slowing and/or an increased accumulated pause time duration at follow-up, this test result forms the basis for further evaluation of the patient, as this slowing of processing speed may suggest a beginning brain degenerative or subcortical brain disorder.

Embodiments of the invention do not comprise cognitive content questions, and thus the above mentioned drawbacks related thereto are avoided. When naming tests are performed in embodiments, these are provided content-independent.

Diseases or conditions to be diagnosed by embodiments of the invention comprise any structural or functional disruption of the cerebrovascular bed, either associated with the normal aging process, or associated with any brain disorder of cortical neurodegenerative or brain white matter origin. Furthermore, this includes any induction of inflammatory processes affecting the blood-brain barrier functions of the brain microvascular system, including any genetic risk factors or genetic polymorphisms associated with these processes. Specific diseases associated with mentioned processes, wholly or in part, include: Alzheimer's disease, Multiple, sclerosis (MS) or any other sub-cortical white matter disease or demyelinating disease, HIV, malaria, cerebrovascular disease (VaD), encephalitis, traumatic brain injury (TBI), mild cognitive impairment (MCI), fronto-temporal dementia (FTD/FLD), dementia with Lewy body disease (LBD/DLB), and Parkinson's disease (PD).

Language in the context of the present application is to be understood as a system for expression of thoughts, feelings etc. by use of a burst of spoken sounds. The use of such a system is a distinguishing characteristic of man compared with other animals. Different nations or people use different languages, e.g. French, Chinese, etc. Two or more individuals speaking the same language can communicate with each other via that system. Language in the context of the present application does expressly not include other systematic or nonsystematic means of communicating, such as gestures or animal sounds.

Speech in the context of the present application is to be understood as the act of speaking, i.e. an utterance of the above mentioned spoken words, independent of a language. Speech in the context of the present application does expressly not include the meaning of national or regional language or dialect. Lungs and vocal cords produce basic sounds that result in speech being produced in a manner of articulation determined how tongue, lips, and other speech organs are involved in making a sound make contact. Speech also comprises pause components of silence or absence of sounds, e.g. between words or sentences.

Prior art systems or methods involving pause components for an analysis in some way are in fact known, and for instance disclosed in U.S. Pat. No. 4,543,957; U.S. Pat. No. 7,272,559; WO 2004/030532; Thomas, C. et. al.: “Automatic detection and rating of dementia of Alzheimer type through lexical analysis of spontaneous speech”, Proceedings of the IEEE International Conference on Mechatronics & Automation, Niagara Falls, Canada, July 2005, Vol. 3, s. 1569-1574; Rosen, K. M. et. al.: “Examining the effects of Multiple Sclerosis on speech production: Does phonetic structure matter?”, Journal of Communication Disorders, March 2007 (in press). None of these disclosures does however use an accumulated pause time of speech in any way.

In U.S. Pat. No. 4,543,957 an apparatus and method are disclosed for diagnosing depression. A dialogue with fluency is held and a response pattern of hesitation pauses in the voice of the subject are measured and classified. Pauses less than 1 second of length are disregarded. Fluency and a response pattern is dependent of the subject's educational and cultural background.

In U.S. Pat. No. 7,272,559 neuro diseases are analyzed. The pronunciation (envelope of registered voice signals) of words is analyzed from a standard sentence read by the subject. Pronunciation of words are highly dependent on the subject's educational and cultural background. Further, only the voice component is analyzed. Pause times are disregarded.

Psychiatric disorders are assessed in the disclosure of WO 2004/030532. Speech cues captured from a patient are analyzed for information in the speech, e.g. a frequency of words is determined, which is highly dependent on the subject's educational and cultural background. Pause times are not considered.

Likewise, in Thomas et. Al a lexical analysis of speech is disclosed. A frequency of usage of different words are analyzed. Pauses are disregarded.

Further, in Rosen et. al. pause times are removed before analysis. Only phonetic content is analyzed, regardless of pauses.

All prior art systems have in common that they are dependent on the educational, social and/or cultural factors, including language, of a subject.

A reliable diagnosis of brain damage induced brain dysfunctions has hitherto not been feasible with systems from the prior art, which is a major drawback, at least with regard to flexibility of the systems for use with different subjects, as mentioned above in the background section.

It is pointed out that the aspects of the invention do not rely on measuring and analyzing single time intervals between spoken words of a subject. Also, pause frequency is disregarded.

Furthermore, the total length of speech is not predefined. This provides for a patient-convenient testing environment without stress. There is no pre-defined time limit for a certain naming task for which total pause time is determined in relation to total speech time. Rather the task is fixed, but not the time for the task. All voice information from an entire measurement period is made use of. Measurement time starts e.g. when stimuli are presented and stopped when the subject so indicates, e.g. by pressing a stop button when a naming task is finished or the subject aborts the speech registration of other reasons (e.g. tired).

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a flow chart illustrating an embodiment of a method;

FIG. 2 is a schematic illustration of an embodiment of an apparatus;

FIG. 3 is a graph showing an excerpt from a registered speech signal of a subject;

FIG. 4 is a graph showing Receiver operating characteristic (ROC) curves of the power of naming speed measures;

FIG. 5 is a schematic illustration showing locations of various regions of interest (ROIs) in the right and left hemispheres of a brain;

FIG. 6 is a color and naming chart for a color and form naming sequence test; and

FIG. 7 is a graph showing relationship between increased level of folate (y-axis in additional % above normal) and the total (accumulated) pause time duration (x-axis in seconds per minute speech).

### DETAILED DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The following description focuses on an embodiment of the present invention applicable to the aging brain and its diseases.

Information processing speed is reduced in many disorders affecting the brain. Processing speed of the verbal output in rapid naming and/or reading can be analyzed by separating the compartments of articulation time and pause time duration. It has recently been shown that processing speed is decreased in the presence of subclincial and/or clinically detectable white matter abnormalities in the brain. Applicants have shown (Warkentin et al., 2008 and see further below) that the reduction in verbal processing speed in Alzheimer's disease is associated with cortical blood flow pathology and that this association is best characterized by an increased accumulated pause time duration. Based on the high sensitivity and specificity of this accumulated pause time duration, a sign for brain dysfunction of subcortical and/or cerebrovascular origin, embodiments of this invention is to serve as a diagnostic tool for such brain dysfunction or the risk, for such brain dysfunction instance in the healthy elderly. In the primary health care the invention may be used in the assessment of probable or possible dementia, and this invention may also be used in self-assessment by subjects who wish to measure their pause time duration and changes thereof after physical exercise, nutritional supplementation, or mental training and/or in research protocols using pharmaceutical and other intervention strategies, aimed to alleviate dementia and dementia related symptoms.

The association between the accumulated total pause time durations in naming or reading tasks and herein mentioned brain dysfunctions has not been described before.

Accumulated or total pause time is here defined as a characteristic of verbal output, produced by any language and during the performance of any cognitive test aimed to measure processing speed. Although pause time assessment (silence) has been described in present technologies, this speech component has been assessed by specific cognitive tasks (for example rapid automatized naming), but the accumulated pause component of random speech has never been used with the above mentioned application.

The pause time duration, described in embodiments of this method, is defined as the total accumulative pause time durations of any length, which are obtained between all of the vocal bursts recorded. The present invention takes advantage of the silent speech component which appears universal in any language.

Moreover, the presented invention is devised not to show dependency educational and cultural factors, including language, of the subject. The language independency is defined as invariant to guttural sound and other types of sound formation which together comprises the sound of speech of an arbitrary language.

### Method

A method for assessment of a brain status of a subject is now described; wherein the brain status comprises a brain damage induced by a brain disease. The method comprises analyzing speech of the subject and determining a pause component of the speech; and determining an occurrence and/or stage of the brain damage induced by the brain disease in the subject based on the pause component. The pause component, absence of sound, is a key component of the present invention. By determining the total (accumulated) pause time duration of a total duration of speech, adding together all recorded individual pause components during a registration, facilitates an information carrier that is more easily investigated than previously.

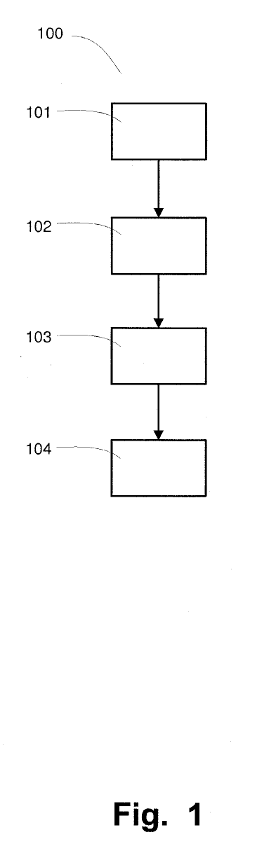

In more detail, in an embodiment of the invention according to FIG. 1 a method 100 is illustrated.

The method 100 comprises a number of steps 101-104. 101: An untimed training session is performed, whereby the subject is accustomed to the name of four colors and four shapes, and their combinations. In contrast to the unequivocal names of the colors, the subject defines the names of the shapes on its own. This procedure is used to avoid the influence of memory and allow for automaticity in the naming fluency. In other embodiments, different parameters and/or numbers thereof may be used instead of four colors and four shapes.

The color stimuli may be shown on a screen.102. A plate with different shapes (e.g. 40 exemplars, but not limited to this number) is presented to the subject. The subject is asked to name the stimuli as quickly as possible, row by row to the end of the plate. The color is named first, then the shape. The voice recording starts when the subject presses a start button and begins to name the stimuli, and ends when the subject presses a stop button after said subject has named the last stimulus on that particular plate. By using a computer and a digital random re-ordering of the stimulus order and the thus obtained random sequence of color and shape combinations occurs each time a suitable stimuli presenting program is started by said subject. This procedure eliminates any effects of learning and memory of the order of presentations of the stimuli or their combinations.

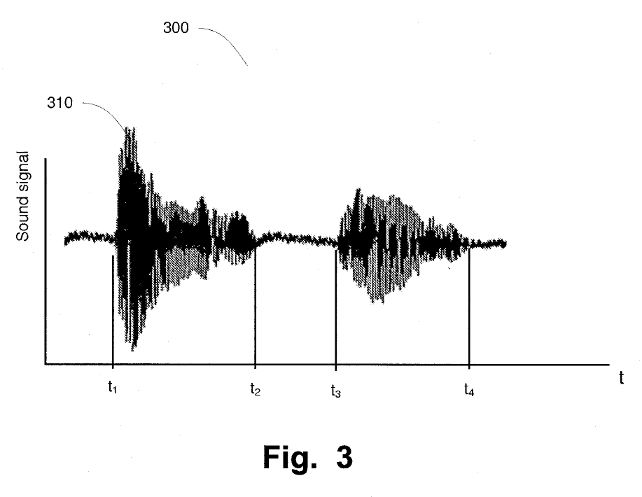

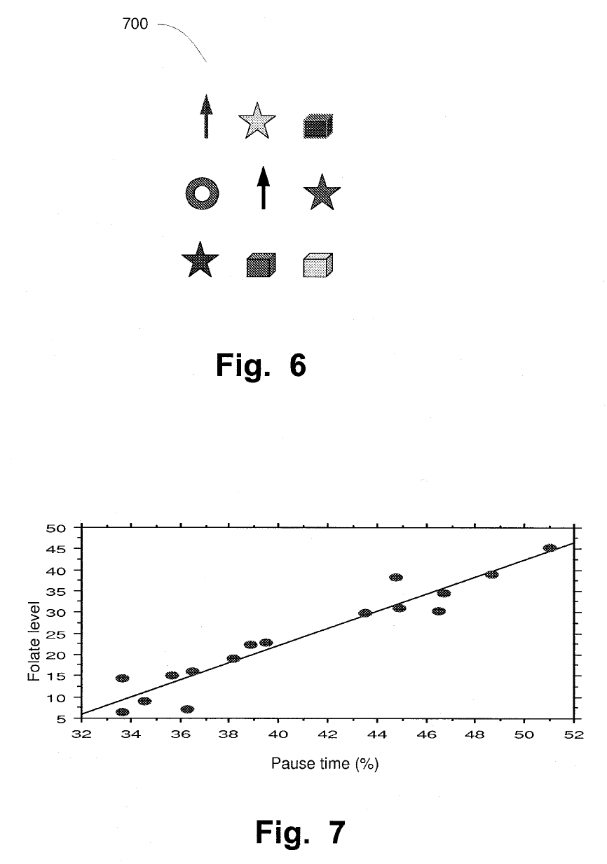

103. The voice recordings are stored in the memory of an embodiment of an apparatus of the present system, e.g. a handheld recording device. Pause and articulation compartments of the voice recordings are automatically analyzed. The accumulated duration of all the pause times of any length (milliseconds) is assessed and measured in relation to the total duration of the naming time (milliseconds) of each particular and randomly generated stimulus set. The duration of total naming time and total pause duration is compared with normal reference values for a diagnosis. The pause time duration, which are obtained between all of the vocal bursts recorded during the overt naming of a randomly generated order and a random order of any number of combinations of different colors and different shapes, e.g. four colors and four shapes. One example of a randomly generated set of stimuli 700 is presented in FIG. 6, and one excerpt of a recorded time series showing several exemplars of pause durations, is shown in FIG. 3.

Instead of a naming task, random speech may be registered and analyzed in other embodiments.

Correlations between the total, accumulated pause time and brain damage induced brain dysfunctions are analyzed for a diagnosis of presence or absence of the dysfunction.

Embodiments of the apparatus allow for the calculation of several indexes on the relation between pause-total time (percent, seconds), and/or pause-articulation time (percent, seconds). Naming errors are not automatically recorded. The reason for this is that naming errors (misnaming of the stimulus, change of naming order) not significantly contribute to the total naming time. In embodiments pause time includes the accumulated inter word pause times between vocal bursts of overt articulation.

Information processing speed (i.e. mental speed) is measured by several tests, but the definition of what is actually measured by these test instruments varies. This means that one and the same tests (for example Digit Symbol, or Stroop Color-Word test, Trail making test, etc.) is interpreted as measuring mental speed in one study, while in other studies the same test is assumed to measure mental flexibility. This is a frequently occurring issue of definition and face validity of test instruments. Reaction time is often used as a measure of psychomotor speed or processing speed. However, this is meant by processing speed within the scope of the present specification. As will be explained in more detail below, there are several different measures of processing speed, comprising decision speed, perceptual speed, psychomotor speed, reaction time, and psychophysical speed. These different components are included in the “pause time” (i.e. preparation and information processing) that is measured. Empirical evidence shows that articulation and pause time are two separate components of the mental processes subserving serial naming tasks, and these two components are not correlated with each other when a verbal response is measured.

### System and Apparatus

In order to perform the test, some embodiments of an apparatus to perform the above describe method comprise a computer, a microphone, and a speech analysis system. These components may be incorporated into a hand-held computer device which is easy to use, and which calculates different components of articulation time, pause time, and various indexes based on the total naming time. Alternatively, a medical workstation may be used for performing the test.

Analyzed parameters may be automatically compared with age-matched normal reference values. An alternative solution may be to measure the total naming time.

Such an apparatus is provided in a system that is devised for assessment of a brain status of a subject, and wherein the brain status comprises the risk for and/or the presence of a brain disease induced brain dysfunction. The system is adapted to determine an occurrence and/or stage of the brain dysfunction induced by the brain disease in the subject. The system comprises an apparatus that is adapted to determine the occurrence and/or presence of the brain dysfunction induced by the brain disease in the subject from the accumulated pause durations between speech sounds produced by the subject. The apparatus comprises units that are operatively connected to each other, which comprises a unit for registering the speech of the subject over a period of time; a unit devised for analyzing the registered speech and configured to determine a pause component of the speech; and a unit that is adapted to determine the occurrence and/or stage of the brain dysfunction induced by the brain disease from the pause component.

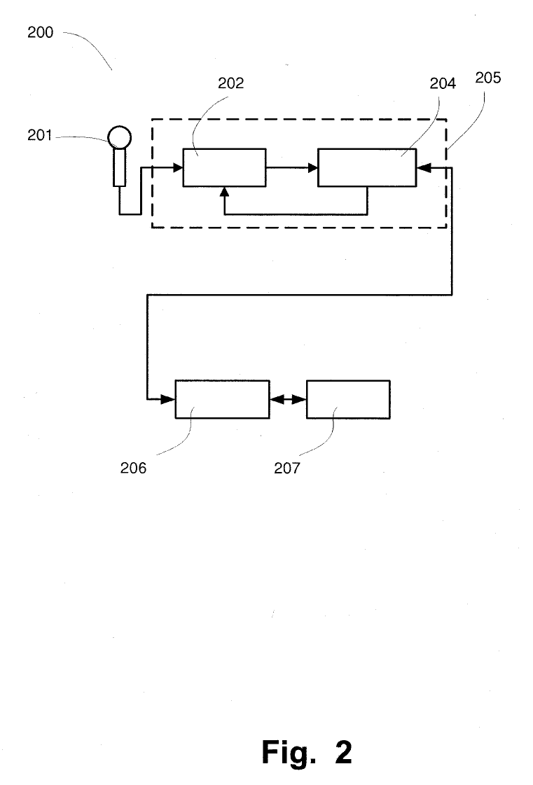

In more detail, FIG. 2 is a schematic illustration of an embodiment of such an apparatus 200 and FIG. 3 is a graph 300 showing an excerpt from a registered speech signal 310 of a subject, with several pauses between vocal bursts.

Apparatus 200 comprises a microphone 201 for registering speech of a subject. The microphone 201 may be any known microphone suitable for registering voice signals and converting these to electrical signals for further processing in the apparatus 200. The microphone 201 is compatible with subsequent processing units, such as Digital Signal Processing (DSP) units, Analog Digital (A/D) converters, processing units, etc. Unit 202 may digitize the signal from the microphone 201 and/or apply a gain control. The converted and/or adjusted signal is then provided to a processing unit 204, which may be a control and sound processing unit.

Units 202 and 204 may be provided as a DSP subsystem that is commercially available. The DSP system communicates with an analyzing unit 206. DSP system 206 may also comprise a memory 207 for, at least temporary, storing or recording the registered speech. The analyzing unit 206 may determine a pause component of the speech, e.g. from a stored speech signal. An example is given in FIG. 3, where a sound signal 310, corresponding to the vocal bursts of speech of a subject, comprises two exemplary words uttered by the subject between times t1 and t2, as well as between times t3 and t4. A pause time is given between times t2 and t3.

The analyzing unit 206 may further calculate indexes; compare calculated results with normal reference values, etc. These indexes may be based on statistical analysis of multiple pause times as the single pause time shown in FIG. 3.

The apparatus 200 further comprises a human user interface for showing the results of the test and communicating with the user.

Some of the embodiments of the present invention may constitute a hand-held device. The hand-held device may in addition comprise an internal microphone or capability for a microphone which may be connected by wire or wire-less, e.g. by Blue Tooth, IR or any other transmission means.

Some of the embodiments may be a software implementation to be executed on a workstation, e.g. computer, laptop. Moreover, some embodiments may additionally comprise a hardware integrated chip with the system integrated to be connected to the workstation, computer or laptop.

The analyzing unit 206 may be comprised in other processing units of the apparatus. Likewise memory 207 may be part of other memory units of the apparatus.

Further embodiments may comprise a USB dongle, (Universal serial bus), to be connected to the workstation, computer or laptop from which the system is executed as a software code or to unlock the system.

### Biological Correlates of Brain Processing Speed

Numerous pathogenic processes are involved in the degeneration of neurons in primary degenerative dementias, such as Alzheimer's disease (AD), frontotemporal dementia (FTD), Parkinson's disease (PD), Lewy-body dementia (LBD/DLB), Amyotrophic Lateral Sclerosis (ALS), and Huntington's disease (HD). Age is the highest risk factor for AD, followed by an overrepresentation of the genetic risk factor ApoE4 ε allele. In addition to this, cerebrovascular pathology within cortical as well as subcortical areas is commonly reported in 60-70% of the AD-cases. Thus, AD shares many of the pathological features seen in vascular dementia (VaD) with the affection of small and large vessels of the brain. Recent evidence has also shown that subjects with mild cognitive impairment (MCI) show subclinical changes of white matter abnormalities, which may constitute risk factor for later development of AD.

Of particular importance in discussions of neuronal versus vessel dysfunction in dementia, are the inflammatory reactions of the vascular endothelial cells. In the brain, these cells constitute the blood-brain-barrier (BBB) and are extremely active in their role to protect the brain from foreign substances in the blood circulation to enter the brain parenchyma. In the presence of stimuli, cascades of molecular events are involved in the inflammatory response to such stimuli. This process involves (among others) the expression of various signalling molecules, and prolonged immunoreactive activation of vascular endothelial cells results in damage of their morphology and function, which (among others) results in an opening of tight junctions and thereby leakage across the BBB. The activation of endothelial cell receptors may also lead to autoimmune diseases such as multiple sclerosis (MS). Although many of the biochemical processes involved in primary dementia and in autoimmune diseases are largely unknown, they do involve specific receptors in cell membranes which activate apoptotic processes (such as for example tumor necrosis factor (TNFα) via the death receptor TNFR1 activating the caspase-pathways), which, among others, lead to the destruction of the myelin-sheets surrounding axonal processes.

Dysfunctional or activated vascular endothelium is a common denominator of many diverse diseases affecting not only the brain (malaria, encephalitis, HIV) but also other bodily organs (such as lungs in chronic obstructive lung disease (COL), liver disease, and heart disease). The build-up of atherosclerotic plaques in the walls of vessels not only severely affects the supply of nutrient and oxygen to organs but also diminished the brain's capacity to rid itself of toxic by-products of cell metabolism, such as soluble or insoluble beta-amyloid (Aβ), as seen in AD.

Experimental evidence has shown that increased levels of plasma homocysteine (destructive for vascular endothelial cell function). Folate together with vitamin B₁₂ counteract the formation of homocysteine and are essential for the methylation processes necessary for all aspects of cell biology, including DNA-methylation. As these vitamins cannot be synthesized de novo by the body, they need to be supplied by food. In the brain, the vitamins are taken up via endocytos by specific receptors on vascular endothelial cells of the blood-brain barrier and by the blood-CSF barrier of the Choroid plexus, and actively transported into the brain parenchyma. Reduced uptake of these vitamins into the cells will affect normal cell function. The polymorphism of the transcobalamin receptors necessary for the uptake of cobalamin (B₁₂) is significantly associated with the level of cerebral blood flow in normal elderly. Thus, genetic predisposition of a reduced ability of vitamin up-take into the CNS (central nervous system) is associated with lower blood flow level in the brain. This relative hypoaemia may contribute in the aging brain to trigger endothelial cell activation, and thereby induce a cascade of events, some of which are deleterious to nerve cells and hence cognitive function.

This may be taken advantage of in systems and methods for determining a level of dependency in a subject of vitamin uptake via determination of increased pause time duration, as described herein.

Vascular endothelium together with vascular smooth muscles cells also regulates the haemodynamic properties of the vessel. The applicants of the present application have recently shown (Janciauskiene et al., 2008) that pro-inflammatory markers for brain vascular endothelial cell activation are associated with lower cerebral blood flow of brain parietal areas in healthy elderly. Thus, higher levels of the vasocontrictor angiotensin converting enzyme (ACE) is associated with higher levels of soluble intracellular adhesion molecule (sICAM-1). The findings suggest that pro-inflammatory processes occur in the vascular bed of the aging brain before clinical signs of cognitive dysfunction. Among the vasodilatory and vasoconstrictive mediators, potassium also acts as a vasodilator. The relation between extracellular potassium level and the genetic risk factor for dementia (ApoE4) was recently investigated by the applicants of the present application in normal healthy elderly. The results showed that ApoE4-carriers had significantly higher plasma potassium values compared with non-carriers. This finding suggests that potassium channels function may be suboptimal in ApoE4-carriers, and that ApoE4-carriers therefore may have a reduced capacity to vasodilate. Therefore, the evidence may suggest that the inverse link between cerebral blood flow and pause time defined herein, is associated with biochemical markers for endothelial cell activation (vasoconstriction, pro-inflammation) and/or genetically determined suboptimal membrane function detectable already in normal aging.

Taken together, any abnormal disruption of the vital function of the brain vascular endothelium will inevitably lead to consequences on the integrity of neuronal and white matter function.

It has been suggested that one of the earliest behavioural consequences of the above mentioned processes might be a diminished speed of the brain to process information. In addition, traumatic brain injury (TBI), effects of street drugs, alcohol abuse or side effects of prescription drugs may also seriously decrease information processing speed of the brain. Not only in all of these instances, but also in the evaluation of the behavioural effects of brain processing speed in relation to pharmaceutical drug treatments (CNS, heart, liver, lung or otherwise), the present invention can be used. Brain dysfunction caused by such causes may thus be determined from the pause time as described herein.

The above biological correlates of brain processing speed are taken advantage of in embodiments of the invention, e.g. in method 100 or apparatus 200.

### Diseases or Conditions to be Diagnosed by Embodiments of the Invention

In general, any structural or functional disruption of the cerebrovascular bed, either associated with the normal aging process, or associated with any brain disorder of cortical neurodegenerative or brain white matter origin may be assessed in embodiments of the invention, e.g. in method 100 or apparatus 200.

This includes any induction of inflammatory processes affecting the blood-brain barrier functions of the brain microvascular system, including any genetic risk factors or genetic polymorphisms associated with these processes.

Specific diseases to be assessed include dementia, such as Alzheimer's disease; Multiple sclerosis (MS); dementia with Lewy bodies (DLB/LBD); Parkinson's disease (PD), Amyotrophic Lateral Sclerosis (ALS) or any subcortical white matter disease or demyelinating disease, HIV, malaria, cerebrovascular disease (VaD), encephalitis, traumatic brain injury (TBI), and mild cognitive impairment (MCI).

These brain disease induced brain dysfunctions are not to be mixed, interpreted or related with mental processes, psychiatric disorders, such as psychoses, including e.g. psychiatric illnesses such as schizophrenia and bipolar disorder, which are not brain disease induced in the sense discussed herein. In psychiatric disorders e.g. the nerve cells of the brain may be intact but the interconnected cells of excitatory and/or inhibitory cells may be dysfunctional due to neurodevelopmental disorders. Assessment of psychiatric disorders may also affect interword pause time, but not in the same manner and not to the same extent as with brain disease induced dysfunctions. Assessment of psychiatric disorders is excluded from embodiments of the present invention.

### Theory Behind Disease Mechanism

Converging evidence shows that decreased processing speed (i.e. perceptual and cognitive slowing) is a behavioral sequelae of a variety of brain disorders. A decreased ability of the brain to quickly process information has been reported in multiple sclerosis (DeLuca at al., 2004), subcortical white matter disease (Junque et al., 1990; De Groot, 2000), subcortical ischemic cerebral vascular lesions (Peters et al., 2005), small-vessel disease (Prins et al., 2005), and Parkinsons disease (Grossman et al., 2002). Processing speed is also reduced in dementia such as Alzheimer's disease (Nebes & Madden, 1988; Nebes et al., 1998), a dementia which is frequently associated with cerebrovascular abnormalities (Agüero-Torres et al., 2006; de la Torre, 1999; Launer, 2002).

The evidence therefore suggests that decreased speed of information processing is seen in brain disorders in which a cortical and/or subcortical cerebrovascular dysfunction is involved in the disease process.

In addressing the putative role of pro-inflammatory markers for brain vascular endothelial activation, the applicant of the present application showed that the level of several adhesion molecules (sICAM-1) and angiotensin-converting enzyme (ACE) were significantly associated with lower blood flow (rCBF) in cortical parietal areas within both hemispheres (Janciauskiene et al., 2008). These findings were obtained while subjects were performing an information processing speed task. Information processing speed may be assessed by continuous naming of simple stimuli (Neuhaus et al., 2001). However, it is not known previously to use standardized and randomly generated continuous naming tasks, for the assessment of processing speed in the aging brain and its diseases, by making use of accumulated vocal bursts and intermittent pause time duration of randomly ordered stimuli, defined herein.

It has been suggested that these two speech compartments reflect different cognitive processes (Hulme et al., 1999).

Of interest is the evidence that pause time duration reflects developmental aspects of the central nervous system (CNS), as the pause time component of naming and reading decreases with CNS-maturation during childhood (Georgiou et al., 2006), while articulation time does not.

The fact that pause time duration is developmentally sensitive and primarily explained by the maturation of brain white matter tracts and its vascular supply, may be taken advantage of in that an age-related or disease-stricken affection of cortical temporal-parietal areas of the brain will inevitably lead to increased pause time durations in cognition (Warkentin et al., 2008).

Hence, processing speed is the most sensitive measure of early CNS-functional disturbance in the aging brain. In fact, several longitudinal studies on normal aging have suggested that a slowing of processing speed in the earliest cognitive sign in those subjects who run the risk of later developing MCI or AD.

As the length of pause time duration is a “pure” estimate of the duration of the cognitive processes underlying naming (Warkentin et al., 2008), any disturbance of these processes (i.e. memory, attention, etc.) will invariably lead to an accumulation and increase in longer pause time durations.

Information processing speed is used as a general term for a number of different types of variables, comprising decision speed, perceptual speed, psychomotor speed, reaction time, and psychophysical speed (Salthouse, 1985, 2000).

Processing speed has often been assessed by means of controlled serial or rapid automatized naming (RAN) tasks (Denckla and Rudel, 1974). From these and other studies it is known know that processing speed becomes slower with increasing age (Perry & Hodges, 1999; Salthouse, 1996). This is also supported by meta-analyses showing a strong relation between normal aging and different speed variables (Verhaeghen and Salthouse, 1997). Pause time duration deviates from these findings by the fact that this speech measure is unrelated to aging. In contrast, articulation time does increase with age. The age-related increase of this particular speech compartment could therefore explain the general slowing of processing speed in naming measures.

FIG. 7 is an illustration of some examples of color and shape combinations of an incomplete set of such combinations. In the example four different shapes are shown. The shapes may have any of four different colors (e.g. black, red, yellow, blue). The method uses either a larger predefined set of such combinations or an undefined set of such combinations, all of which may be randomly generated and randomly ordered by the method. Such a chart may be provided virtually via a user interface, e.g. of a medical workstation, to the subject to be tested.

Table 0 gives further statistical data showing that pause time is unaffected by age but is increased in dementia. Means and standard deviations for pause times (index a in the table) are given. No statistically significant differences are seen between the age intervals with each subject group (Bonferroni corrected). Comparisons between normal subjects (upper part of table 0) and Alzheimer patients (lower part of table 0) reveal a factorial ANOVA for pause time (percent, %), F=26.408, df. 98, p<0.0001.

TABLE 0

| Age interval (years) | 50-60 | 61-70 | 71-80 | ≧81 |
|---|---|---|---|---|
| Pause time | 46.0 (10.0) | 41.9 (9.8) | 43.7 (6.3) | 41.2 (5.8) |
| (percent, %) | | | | |

Alzheimer's disease (age range 59-89 years)

| | | | | |
|---|---|---|---|---|
| Pause time | 54.6 (9.9) | 57.4 (11.9) | 56.5 (7.5) | 50.1 (7.8) |
| (percent %) | | | | |

The above shown cut-off values (Table 0) may be applied in some embodiments for assessing a brain status of a subject by thresholding analyzed pause times of speech of the subject, wherein said brain status comprises a brain disease induced brain dysfunction:

a) Healthy: Pause time in percent less than or equal to approximately 50% of the total time used by the subject to name a predefined set of colour and shape combinations, e.g. 49%, 45%, 40% or less:

The subject is healthy with regard to the brain disease induced brain dysfunction (no occurrence of brain disease induced brain dysfunction in the subject)

b) Pathologic: Pause time, in percent the total time used by the subject to name a predefined set of colour and shape combinations, is longer than approximately 50-60%, e.g. longer than 50%, or longer than 60%, e.g. 55%, 65%, 75%:

The Subject is at risk for suffering from a brain disease induced brain dysfunction and further investigation by professional health care facilities is recommended.

The ranges of pause time duration based thresholds may be used advantageously for the assessment of subjects in embodiments of the invention.

In the below example, empirical evidence is given showing that serial verbal responses in continuous naming can be separated into two compartments, i.e. articulation and pause time.

As previously stated, one pertinent aspect is that articulation and pause time are not significantly related. This dissociation has been suggested to reflect independent storage and retrieval processes (Hulme et al., 1999). The independent nature of these two speech compartments has also been demonstrated in brain development (Georgiou et al., 2006), during which pause time decreases in maturing children while articulation is not affected. Thus, pause time is developmentally sensitive, whereas articulation is not.

In a recent fMRI-study, Kircher and coworkers (2004), demonstrated that articulation during continuous speech engaged different brain areas than did pause time. The authors suggested that normal pause duration reflects speech planning, and in particular lexical retrieval.

On the basis of these findings, the applicants of the present invention draw the inventive conclusion that it is reasonable to expect that pause time and articulation time should also be differentially affected in brain dysfunctions induced by diseases, such as dementia, including Alzheimer's disease, especially as memory retrieval difficulty is an important clinical symptom of such diseases.

In particular, these two speech compartments could hypothetically be differentially associated with the typical temporo-parietal rCBF pathology reported in Alzheimer's disease (Risberg & Gustafson, 1997; Hock et al., 1997; Mentis et al., 1996).

Perfusion deficits in Alzheimer's disease are also evident by an inability of patients to activate cortical areas in response to cognitive tasks, such as verbal fluency (Warkentin & Passant, 1997).

Cortical inactivation has also been demonstrated in several fMRI-studies in Alzheimer patients, but inconsistent findings have also been reported (Bäckman et al., 1999; Trollor et al., 2006; Woodard et al., 1998).

Decreased perfusion in the brain of Alzheimer patients has been suggested to reflect an impaired neurovascular autoregulation (Girouard & Iadecola, 2006; Iadecola, 2004), and long-term hypoperfusion in Alzheimer's disease is thought to promote ischemic lesions in cortical as well as subcortical areas (Brun & Englund, 1986).

However, although many studies have reported on the cognitive sequelae of the rCBF-pathology in Alzheimer's disease, specific associations between brain perfusion deficits and processing speed have so far not been shown, or investigated in this dementia.

Based on the findings of a dissociation of speech measures discussed above, the hypothesis that not only a general slowing of processing speed, but in particular pause time, is the behavioural output measure which most closely relates to cerebrovascular dysfunction of Alzheimer's disease, has been empirically proven in the example study described below.

Some embodiments of the invention are implemented in a medical workstation. The medical workstation comprises the usual computer components like a central processing unit (CPU), memory, interfaces, etc. Moreover, it is equipped with appropriate software for processing sound data received from sound data input sources, such as data obtained from microphone devices.

A computer program for processing by a computer is provided is some embodiments. The computer program is configured for assessment of a brain status of a subject, wherein the brain status comprises a brain damage induced by a brain disease. The computer program comprises a first code segment for analyzing speech of the subject and determining a pause component of the speech; and a second code segment for determining an occurrence and/or stage of the brain damage induced by brain disease in the subject based on the pause component.

The computer program may for instance be stored on a computer readable medium, accessible by the medical workstation.

The medical workstation may further comprise a monitor, for instance for the display of rendered visualizations, as well as suitable human interface devices, like a keyboard, mouse, etc., e.g. for interacting with the medical workstation. The medical workstation may be part of a system. The medical workstation may also provide data for suggesting treatments based on the assessment outcome. The medical workstation may have a graphical user interface for computer-based assessment of brain damage induces brain dysfunctions. The graphical user interface may comprise components for visualizing the methods described above in this specification or recited in the attached claims.

Embodiments of the system or apparatus described herein may advantageously be implemented and used for carrying out a method, such as the above described or the following method.

A method for assessment of a brain status of a subject, wherein the brain status comprises a brain disease induced brain dysfunction, wherein the method comprises analyzing speech of the subject and determining a pause component of the speech, as defined herein; and determining an occurrence and/or stage of the brain disease induced brain dysfunction in the subject based on the accumulated pause duration times.

The method may comprise registering the speech and/or recording the speech of the subject over a period of time; and wherein the analysis of the speech comprises the analysis of the registered speech and/or the recorded speech for determining the length of the pause component between vocal bursts of the speech.

In the method the analyzing the overt speech of the subject may be performed irrespective of a language of the speech.

The method may comprise applying a compensation factor for a specific language of the speech for the assessment.

The method may comprise applying a compensation factor related to an age of the subject.

In the method the assessment may be a cognitive test based assessment, comprising the subject freely defining parameters of the cognitive test.

The method may comprise providing a basis for medical personal for deciding if a subject has signs of a brain disease induced brain dysfunction or not.

The method may comprise directing primary health care resources to those subjects who are at high risk for having a brain disease induced brain dysfunction, and who need further assessment for their diagnosis, while saving financial costs for unnecessary evaluations of patients with negative test results.

The method may comprise basing the occurrence and/or stage of the brain disease induced brain dysfunction on a threshold value of the accumulated pause time component.

In the method the threshold value may comprise different ranges for the occurrence and/or stage of the brain disease induced brain dysfunction, and the methods comprises determining a) an accumulated duration of pause time less than or equal to approximately 50% for a healthy subject; b) an accumulated duration of pause time between approximately 50% to 60% for a subject at risk for or in an early stage of the brain disease induced brain dysfunction.

The method may comprise a cognitive test performed by the subject, wherein the pause time component comprises a mean duration of the accumulated pause times measured in relation to the total duration of a naming time of the cognitive test performed by the subject. The aforementioned threshold value refers to such cognitive tests.

The method may further comprise determining the occurrence and/or stage of the brain disease induced brain dysfunction by comparing the total duration of the total naming time and a total accumulated pause duration with normal reference values.

The method may comprise determining the occurrence and/or stage of the brain disease induced brain dysfunction from the accumulated pause component by calculating at least one index on the relation between total accumulated pause duration, pause-articulation time, in percent or in seconds.

In the method the determining of the occurrence and/or stage of the brain disease induced brain dysfunction from the pause component does not comprise registering of naming errors.

The method wherein the determining of the occurrence and/or stage of the brain disease induced brain dysfunction from the pause component may comprise associating an increase in accumulated pause times with white matter function/dysfunction and/or cerebrovascular dysfunction, in either healthy aging, mild cognitive impairment (MCI) or dementia.

In the method the assessment may be cognitive test based assessment, wherein the subject is free to define parameters of the cognitive test, wherein the cognitive test provides measures of processing speed, such as for example using simple colors and shapes, or naming other defined stimuli, and is non-invasive.

The method wherein the cognitive test may be implemented in an education and culture-free manner, and wherein the cognitive test does not comprise questions related to knowledge of the subject.

In the method the brain disease induced brain dysfunction may be not of developmental origin of the central nervous system (CNS), but reflects the aging and disease processes of the CNS in the elderly.

The method may further comprise determining the dependency of a subject on adequate vitamin levels via determination of the pause component.

In an example of diagnosis for which some embodiments of diagnostic methods may be provided, is to assess indications of elevated levels of folate in a patient. FIG. 7 is a graph showing the relationship between increased level of folate (y-axis) and the total (accumulated) pause time duration (x-axis) The total pause time duration (percent of total naming time) accumulated during naming of a predefined set of randomly generated color and shape combinations, a subset of which are illustrated in FIG. 6. The reasoning for the occurrence is that Folate levels correlate with total pause time duration obtained during naming of randomly generated color and shape combinations of a predefined set of such combinations in healthy subjects carrying one or two copies of the □4 allele of the apolipoprotein E gene. This example is elucidated in more detail below.

In embodiments of the method the brain disease induced brain dysfunction may be related to dementia, such as Alzheimer's disease; Multiple sclerosis (MS); Parkinson's disease (PD); dementia with Lewy bodies (DLB/LDB); Amytrophic Lateral Sclerosis (ALS); subcortical white matter disease or demyelinating disease; HIV; malaria; cerebrovascular disease (VaD); encephalitis; traumatic brain injury (TBI); mild cognitive impairment (MCI); traumatic brain injury (TBI); effects of street drugs; alcohol abuse; side effects of prescribed drugs and/or pharmaceutical drug treatments; and diseases of other bodily organs such as heart, liver, lung or otherwise.

Also, the system or apparatus may be used for assessing the status of brain disease induced brain dysfunction in a subject, wherein the brain disease induced brain dysfunction is related to dementia, such as Alzheimer's disease; Multiple sclerosis (MS); Parkinson's disease (PD); dementia with Lewy bodies (DLB/LDB); Amytrophic Lateral Sclerosis (ALS); subcortical white matter disease or demyelinating disease; HIV; malaria; cerebrovascular disease (VaD); encephalitis; traumatic brain injury (TBI); mild cognitive impairment (MCI); traumatic brain injury (TBI); effects of street drugs; alcohol abuse; or side effects of prescribed drugs and/or pharmaceutical drug treatments, and diseases of other bodily organs such as heart, liver, lung or otherwise.

The above described computer program may in some embodiments enable carrying out embodiments of the above described method.

### EXAMPLE

Below, an example is given, wherein brain imaging was used to determine information processing speed of the brain and different regions thereof. Accumulated pause time durations and articulation times were examined as input parameters for assessing a degree of a brain damage induced disease, such as dementia, for which in a specific example Alzheimer's disease is investigated.

Decreased information processing speed (mental slowing) is a known sequelae of many brain disorders, and can be assessed by continuous naming tasks. Functional imaging studies have shown that pause and articulation times in continuous speech are normally associated with different brain regions, but knowledge about such association in dementia is lacking. We therefore tested the hypothesis that perfusion deficits in Alzheimer's disease (AD) are not only associated with slower processing, but also with these separate speech measures. Using regional cerebral blood flow (rCBF) measurements during the performance of a continuous color and form naming task, we found that naming speed was substantially slower in AD patients than in controls. This slower naming was exclusively determined by an increase in accumulated pause time, and only to a limited extent by articulation time. The increased accumulated pause time was uniquely associated with temporo-parietal rCBF reductions of the patients, while articulation time was not.

By contrast, the rCBF of healthy elderly control subjects was consistently accompanied by substantially shorter articulation and pause times, although the naming measures were not statistically associated with rCBF.

These findings suggest that an increase in the accumulated pause times (in contrast to articulation time) may serve as the most sensitive measure in the assessment of information processing speed deficits in dementia, by virtue of its close association with brain pathology.

All subjects were native speakers of Swedish, and were predominantly right-handed as measured by the Edingburgh handedness inventory (Oldfield, 1971). All subjects were screened for the absence of any neurological disorder, mental illness and drug or alcohol abuse. Standard laboratory blood tests were all normal in the healthy elderly.

MiniMental Test (MMSE, Folstein et al., 1975) scores were normal for their age and educational level.

Before inclusion of patients with Alzheimer's disease, all patients underwent a thorough clinical investigation including medical history, cognitive testing, neurological examination, laboratory tests, and CT-scan in order to rule out other causes of dementia. The clinical diagnosis of dementia was made by DSM-IV and probable Alzheimer's disease was determined by the exclusion of other dementias in accordance with the NINCDS-ADRDA criteria (McKhann et al., 1984).

Assessment of Processing Speed

We used a simple measure of information processing speed, which comprised of 40 color and shape combination stimuli. Four different colors and four different shapes were combined in a random fashion The standard test procedure started with a short training session, during which the subject was presented with four different colors, four different shapes, and four combinations of these, and was asked to name these stimuli correctly. During this untimed session any errors made by the subject were corrected by the examiner. Thereafter, the subject were asked to name the colors and shapes of the stimulus combinations as quickly as possible. The primary outcome measure was the time (seconds) it took the subjects to name all of the combinations presented in the matrix. Naming errors were recorded when subjects did not self-correct their errors.

In order to investigate which cortical areas of the brain are related to the accumulated pause time duration, regional cerebral blood flow (rCBF) was measured while subjects performed the test.

Cerebral Blood Flow Imaging (rCBF)

The regional cerebral blood flow was measured by the non-invasive 133Xe-inhalation method as described by Obrist et al. (1975) and Risberg et al. (1975). This method gives information about the blood flow in superficial cortical areas only. We used a system with 64 scintillation detectors (NaI (Tl) crystals) arranged in a helmet around the head (Cortexplorer 64, Ceretronix, Denmark). The system adjusts for differences in head size and shapes, and the positioning of the head is standardized in relation to bony landmarks (nasion and ear channels) by means of light crosses. This makes it possible to reposition subjects accurately in case of head movements.

The measurement procedure used in this study was as follows: before the rCBF measurement began, all subjects underwent a short untimed training session of naming the stimuli four colors and forms, and four combination of these, as mentioned above. After this practice session, the rCBF-measurements were performed with the subjects in the supine position and the stimulus matrix (plate) was aligned over the subject's head with best possible visual adjustment. Acoustic recordings were made with a real-time spectrum analyzer (Spectra Plus, 32 bit for Windows, version 2.32, Pioneer Hill Software) using a single channel with fast Fourier transformation. The separation of silent epochs and speech bursts were performed manually by measuring the duration of each separate silent epoch in milliseconds on the time series. The remaining time of the recording represented the articulation time. The intrusion of task irrelevant sounds, (such as coughing for example) were excluded from the analysis.

Statistical Analysis

In order to reduce the possibility of Type 1 and Type 2 errors in multiple comparisons, four regions of interest (ROIs) were selected from the detector array (FIG. 5); two ROIs within each hemisphere, with one ROI located in dorsolateral frontal areas and the other in temporo-parietal areas.

The mean of the normalized values for the detectors included within each ROI was calculated and used in the within- and between-group comparisons and in the comparisons with the naming time measures. Between-group comparisons of rCBF were performed by t-tests for unpaired (two-tailed), as the flow values of the ROIs were normally distributed. Spearman's rank correlations were used to analyze the relation between naming times and rCBF, as well as the relation between the naming measures. Spearman rank correlations were also used to analyze the relation between the rCBF-distribution values of the pooled group and the subject groups separately, in order to investigate the separate relations between rCBF and the total naming time, the accumulated pause time duration, and the articulation times. Receiver operating characteristic (ROC) curves were calculated for between-group differences in the total naming time, pause and articulation time, and the differences between the areas under the curves (AUC) were assessed.

### Results

Naming Speed Measures.

Table 1 shows the mean and standard deviations for the total naming time, articulation time, pause time, the articulation/total time ratio, and the pause time/total time ratio. All statistical comparisons between the normal controls and the patient group were highly significant. Thus, patients had longer total mean naming time, as well as longer mean articulation and accumulated pause time durations than the normal controls. However, the means for articulation and pause times were in opposite directions between the subjects groups. Thus, pause time was significantly longer than articulation time in patients, while the normal controls had a higher mean articulation time than pause time. The same directional difference of the group means of the naming measures was also seen in the proportion of pause time (the ratio between pause time and total naming time in percent) which was significantly higher than articulation time in the patient group, while the opposite was seen in the normal control group were the proportion of articulation time was higher than pause time. The within-group differences between the naming measures were highly significant, suggesting that articulation time and pause time were independent.

TABLE 1
Colour and form naming times (seconds)

| | Normal controls | Patients | |
|---|---|---|---|
| | (n = 57) | (n = 48) | |
| | Mean (SD) | Mean (SD) | P-value ¹ |
| Total time | 52.3 (8.8) | 89.6 (23.5) | 0.0001 |
| Articulation | 29.7 (5.4) ^{a} | 38.7 (6.3) ^{b} | 0.0001 |
| time | | | |
| Pause time | 22.5 (6.2) | 50.9 (21.0) | 0.0001 |

Ratios

| | | | |
|---|---|---|---|
| Articulation time/ | 57.1 (7.6) ^{c} | 44.7 (8.7) ^{d} | 0.0001 |
| Total time (%) | | | |
| Pause time/ | 42.7 (7.5) | 55.2 (8.8) | 0.0001 |
| Total Time (%) | | | |

¹ Comparison between normal controls and patients, unpaired t-test
^{a, b} Within-group comparison of articulation time versus pause time, p < 0.0001, t-test.
^{c, d} Within-group comparison between ratios, p < 0.0001, t-test.

We performed receiver-operating characteristic (ROC) curves on the total naming time, pause time and articulation time, to further illustrate the extent to which the naming time measures discriminated between the normal controls and the patients.

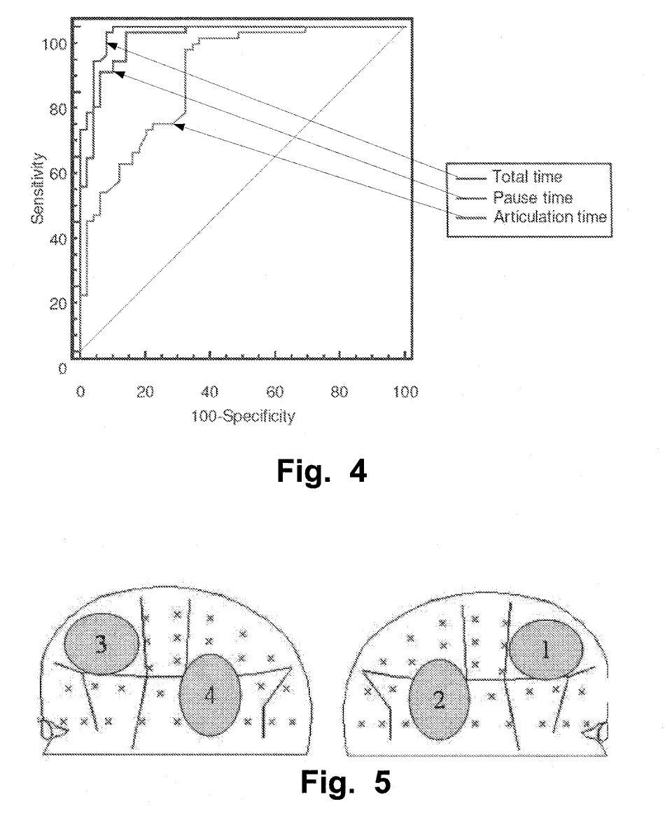

FIG. 4 is a graph showing Receiver operating characteristic (ROC) curves of the power of naming speed measures, to discriminate between Alzheimer patients (n=47) and healthy elderly controls (n=59). The total naming and pause time showed high diagnostic accuracy with 98.4% and 96.3%, respectively, while articulation time showed a modest accuracy of 85% of the area under the ROC curve. The sensitivity and specificity values were for the total naming time 98.3% (95% CI 90.6-99.7) and 91.8% (95%, CI 80.4-97.7), for pause time 98.3% (95% CI 90.6-99.7%) and 85.7 (95% CI 72.7-94.0) and for articulation time 93.0% (95% CI 83.0-98.0) and 67.4% (95% CI 52.5-80.0), respectively.

The AUC was 98.4% for total naming time, 96.3% for pause time, and 85% for articulation time, and the differences between the ROC-curves were significant between articulation and pause time (p<0.005) and between articulation and total time (p<0.001), while pause and total naming time was not significant (p<0.12).

Regional Cerebral Blood Flow (rCBF) Obtained During Naming

The mean hemispheric absolute blood flow values and the expiratory CO2-values are shown in Table 2. The flow values were significantly lower in the patient group than the normal control group. Although the PeCO2 was slightly lower in the patients, this difference was not statistically significant.

TABLE 2
Mean hemispheric CBF obtained during task performance

| | Normal controls | Patients | |
|---|---|---|---|
| | (n = 57) | (n = 49) | |
| | Mean (SD) | Mean (SD) | P-value ^{b} |
| Right hemisphere | 41.6 (4.6) ^{a} | 37.2 (4.9) | 0.0001 |
| Left hemisphere | 41.5 (4.5) | 37.7 (4.8) | 0.0001 |
| PeCO₂ | 34.2 (3.2) | 33.2 (4.0) | NS |

^{a} Uncorrected for PeCO₂
^{b} Unpaired t-test

Table 3 shows the mean distribution normalized rCBF-values of the ROIs between the subject groups. The regional rCBF-differences were highly significant, between the groups. Thus, the patients had significantly higher rCBF-values in dorsolateral frontal areas bilaterally (ROIs 1 and 3), while they had significantly lower values in the temporo-parietal areas (ROIs 2 and 4) than the Controls.

TABLE 3
Normalised rCBF values (%) obtained during task performance

| | Normal controls | Patients | |
|---|---|---|---|
| | (n = 57) | (n = 49) | |
| ROIs | Mean (SD) | Mean (SD) | P-value ^{a} |
| 1 | 98.6 (1.9) ^{a} | 100.4 (3.2) | 0.0006 |
| 2 | 99.6 (1.4) | 98.2 (2.4) | 0.0003 |
| 3 | 98.9 (2.2) | 101.5 (3.6) | 0.0001 |
| 4 | 99.3 (1.4) | 97.3 (2.1) | 0.0001 |

^{a} Unpaired t-test

Comparison Between rCBF and Naming Speed.

Spearman rank correlations were performed between ROIs and the naming speed measures within each groups, as shown in Table 4.

TABLE 4
Table 4 Spearman rank correlations between naming times and normalised rCBF

| | | | | | |
|---|---|---|---|---|---|
| | | | | Articulation/ | Pause time/ |
| ROI | Total time | Articulation time | Pause time | Total time | Total time |

Normal controls (n = 57)

| | | | | | |
|---|---|---|---|---|---|
| 1 | 0.016 | −0.113 | 0.125 | −0.182 | 0.187 |
| 2 | −0.017 | −0.005 | −0.011 | 0.006 | −0.006 |
| 3 | 0.001 | −0.057 | 0.055 | −0.074 | 0.080 |
| 4 | 0.221 | 0.237 | 0.105 | 0.060 | −0.060 |

Patients (n = 49)

| | | | | | |
|---|---|---|---|---|---|
| 1 | 0.167 | 0.059 | 0.170 | −0.200 | 0.202 |
| 2 | −0.264 | 0.249 | −0.223 | 0.142 | −0.147 |
| 3 | 0.316 | 0.094 | 0.327 | −0.303 | 0.303 |
| 4 | −0.472 a, † | 0.029 | −0.521 b, ‡ | 0.504 c, # | −0.506 d, ¶ |

Difference in correlation coefficients between normal controls and patients:
a, z = 3.675, p < 0.0002,
b, z = 3.405, p < 0.0007,
c, z = 2.735, p < 0.007 (trend), and
d, z = 2.479, p < 0.02 (trend).
Correlations between naming times and ROI 4:
† F-test 13.368, p < 0.006,
‡ F-test 17.542, p < 0.001,
# F-test 16.182, p < 0.0002,
¶ F-test 15.997, p < 0.0002.
Bonferroni correction, p < 0.002.

No significant correlation between the ROIs and the naming measures was seen in the normal control subjects. However, in the patient group the total naming time, pause time, and the articulation and pause time ratios were significantly correlated with left temporo-parietal areas (ROI 4), while articulation time was not. In addition, the correlation coefficient for the accumulated pause time duration in this area were significantly different between the normal controls and the patients, suggesting that this naming measure was uniquely associated with the left temporo-parietal raCBF-pathology in the patients.

Naming Errors

The mean naming errors for the normal controls was 0.5 (SD: 0.8, range 0-6 errors) and for the patients 1,3 (SD: 1.8, range 0-3). This difference in error rates was significant (t=3.058, p<0.005). However, the number of errors did not significantly correlate with either the naming measures or with rCBF within the separate subject groups.

Naming Times and Age

Age was not significantly related to any of the naming measures in the pooled group of subjects (after Bonferroni correction, p<0.006) or the Alzheimer group. However, significant correlations were seen in the normal control group, showing that the total naming time increased with age (r=0.48, df: 56, p<0002), as did articulation time (r=0.49, df. 56, p<0,0001), but not the pause time durations.

The results of this study demonstrate that naming speed is substantially slower in patients with Alzheimer's disease than in normal healthy elderly control subjects. This difference was largely determined by significantly longer pause time durations in the patients, and only to a minor degree by articulation time. The longer pause times were also significantly related to temporoparietal rCBF-pathology in the patients, while no such relation was seen in the controls.

Receiver operating characteristic curves showed very high sensitivity and specificity values for the total naming time and the pause time, in the differentiation of patients from normal controls.

Normal Ageing

Regression analyses performed on the normal controls and the patient groups separately, showed that ageing was positively related with the speed measure, but only in the control group and not in the patient group. Thus, the regression coefficient for age and the total naming time (articulation and pause time) in the controls was 0.48 (p<0.0002). The analyses on the two speech compartments separately showed that only the acoustic output time for was age-related (r=0.49, p<0.0001), while pause time was not. These findings confirm that normal aging is associated with a decrease in information processing speed (Salthouse, 2000), but our findings show that this normal age-related slowing is explained mainly by the rate of verbal output (articulation) in healthy aging, not the length of pause time durations.

Naming Speed and Alzheimer's Disease

There were highly significant negative correlations between pause time duration (not articulation time) with left temporo-parietal areas in Alzheimer patients, but no significant correlation was seen in the normal controls. This is an interesting finding in that it not only strengthens the dissociation between these naming measures, but that it specifically shows that the pause time duration is associated with brain areas which are almost invariantly dysfunctional in Alzheimer's disease, often with a left-sided dominance of pathology (Warkentin et al., 2004). In light of the previous discussion that the pause time component of speech may reflect retrieval processes (Hulme et al., 1999; Kircher et al., 2004), the dissociation of naming times seen in the present study, could reflect the patient's difficulties to retrieve the names of the stimuli, despite the repeated performance of the task. In fact, the general impression of listening to the audio-recordings showed that patients did not have any difficulties to name the colors (i.e. had no perceptual difficulties), but instead often showed a marked hesitation when trying to recall the name of the shapes of the stimuli. Thus, difficulties in retrieving the names of shapes seems to be the major aspect of the naming task, which could explain the substantial slowing in naming speed in Alzheimer patients.

Decreased processing speed has been reported in a variety of brain disorders, primarily of subcortical vascular origin, and as vascular factors are linked to the development of Alzheimer's disease (Brun, 2003), further studies are warranted to illuminate the relation between brain hemodynamic reactivity and processing speed in Alzheimer disease.

What Does Decreased Naming Speed Mean in Alzheimer's Disease?

The present findings of an association between decreased processing speed (i.e. increased pause time duration) and decreased blood flow in Alzheimer patients suggests the possibility that processing speed may be associated with early cognitive decline. In fact, this possibility has been implicitly shown in population-based studies of predictive factors for subsequent diagnosis of Alzheimer's disease (the Rotterdam study, Amieva et al., 2000; Fabrigoule et al., 1998). In their analysis of possible preclinical changes of cognitive function, it was demonstrated that not only measures of higher cognitive abilities but also simpler and more general functions, such as processing speed, are important measures for the identification of subtle deterioration in seemingly cognitively intact individuals, who may be at risk for developing dementia. This was also reported in a recent MRI-study (Bartzokis et al, 2007) showing that signs of demyelinisation in subcortical fiber tracts of healthy subjects with genetic risk factor for Alzheimer's disease, was associated with slower cognitive processing speed.

Taken together, the evidence clearly suggests that both cortical and subcortical vascular dysfunction share the same behavioral outcome of cognitive slowing. Our findings support this evidence and further suggest that pause time (in contrast to articulation time) may serve as a sensitive measure in the assessment of information processing speed deficits in dementia, by virtue of its close association with brain pathology.

### Further Example

The applicant of the present application also recently found that decreased information processing speed (i.e. increased pause duration times) is also related to the plasma folate level in the elderly. While the ApoE4-genotype (a genetic risk factor for dementia) also has been associated with decreased processing speed, it was prior to the study, as described below, still unknown whether the observed relation between processing speed and folate was specifically associated with this risk factor.

Participants and Methods: Fifty-four healthy elderly (mean age 72.4, SD 7.4) performed a processing speed naming task (simple color and shape naming). Simultaneous voice-recordings of their verbal response were analyzed by calculating the articulation and pause time durations obtained during naming of a predefined set of stimulus combinations. Fasting plasma folate levels were obtained in the morning before the test session, and apolipoprotein E (ApoE) genotype was determined for each individual.

Results: Spearman rank correlations and regression analyses showed that naming speed and folate was significantly related in ApoE4 carriers (ApoE4+, n=16), but not in non-carriers (ApoE4−, n=38). Thus, a longer mean duration and a higher frequency of the pause times between speech sounds was associated with elevated folate levels (corr. coeff. 0.910, p<0.0001) in ApoE4+, while this was not seen in ApoE4−. The mean articulation time was negatively associated with folate (p<0.0001), suggesting that slower naming of the stimuli (i.e. increased pause time duration) was associated with higher levels of plasma folate. Importantly, the correlation coefficients were significantly different (p<0.01 to p<0.0001) between the ApoE4+/−subgroups, substantiating the specificity of an association between processing speed and plasma folate level in ApoE4 carriers.

### CONCLUSIONS

There is an association between elevated plasma folate levels and decreased processing speed in the aging brain that differs between ApoE4 carriers and non-carriers. These findings strongly suggest that ApoE4 carriers are highly folate-dependent in order to maintain adequate processing speed, while non-carriers are not. Hence, information processing speed is associated with folate in ApoE4+but not in ApoE4−healthy elderly.

This may advantageously be implemented in some embodiments of the invention, wherein pause time related thresholding, such as according to the above mentioned ranges, may be used to identify subjects who's folate uptake is genetically determined.

Further examples, applications and uses in which the present invention may be beneficial are described below.

To assess any training effects on pause time duration, performed by a subject, either by physical training and exercise to improve brain blood flow and brain oxygenation and/or by any mental training programmes which are aimed to improve any cognitive abilities, such as for example memory function and reading and writing abilities, of that subject.

To assess the effects on pause time duration of any nutritional supplementations used by the subject, which supplementation is aimed to improve the physical and/or mental well-being of that subject. Such supplementations may involve any vitamin supplementation and any supplementation of any polyunsaturated fatty acids aimed to improve the lipid metabolism of the brain of that subject.

To assess the effects on pause time duration of any pharmaceutical intervention approach aimed at improving the transmission of any neurotransmitter subservient to any mental processes performed by the brain, such as for example any pharmaceutical drug present or developed in the future for the treatment of dementia disorders. Furthermore, to assess the effect on pause time duration by reducing the build-up of toxic by-products within the brain and/or to increase the elimination of toxic waste products of metabolism in the brain, via the blood-brain barrier and/or via the blood-cerebrospinal fluid barriers of the brain.

To assess the effects on pause time duration of any pharmaceutical and/or genetic intervention approach aimed at influencing or manipulating the cleavage processes by protease inhibitors of the amyloid precursor protein (APP), the protein which is thought to contribute to the build-up and the formation of neurofibrillary tangles and the formation of senile plaques (soluble or insoluble) within the brain parenchyma and the endothelial cells of the blood vessels in the brain, as these processes are thought to be at the core of the cognitive dysfunctions in Alzheimer's disease and vascular dementia.

To assess any effects on pause time duration of any pharmaceutical or genetic approach aimed to improve the symptoms of Parkinson's disease and Parkinson's dementia which affect any neurotransmitter system in the brain which overlaps with those neurotransmitter systems known to degenerate in Alzheimer's disease, dementia with Lewy bodies (also called Lewy body dementia), and Frontotemporal dementia.

To assess the effects on pause time duration of any other disorder than those mentioned earlier, which is known to slow down the brain's ability to process information, such as motorneuron disease, tumor, or stroke.

To assess the effect on pause time duration of any metabolic or otherwise dysfunction in other bodily organs than the brain of a subject, which can effect the cognitive performance of the brain.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

### REFERENCES

The above specification refers to the following references, which are incorporated herein in their entirety for all purposes:

Agüero-Torres, H., Kivipelto, M., & von Strauss, E. (2006). Rethinking the diagnoses in a population-based study: what is Alzheimer's disease and what is vascular dementia? A study from the Kungsholmen project. Dementia and Geriatric Cognitive Disorders, 22, 244-249.

Amieva, H., Rouch-Leroyer, I., Fabrigoule, C., & Dartigues, J-F. (2000). Deterioration of controlled processes in the preclinical phase of dementia: a confirmatory analysis. Dementia and Geriatriatric Cognitive Disorders, 11, 46-52.

Bartzokis, G., Lu, Po. H., Geschwind, D. H., Tingus, K., Huang, D., Mendez, M. F., Edwards, N., & Mintz, J. (2007). Apolipoprotein E affects both myelin breakdown and cognition: Implications for age-related trajectories of decline into dementia. Biological Psychiatry, doi: 10.1016/j.biopsych.2007.03.024.

Brun, A. (2003). Vascular burden of the white matter.

International Psychogeriatrics, 15(1), 53-58.

Brun, A., & Englund, E. A. (1986). A white matter disorder in dementia of Alzheimer type. Annals of Neurology, 19, 253-262.

Bäckman, L., Andersson, J. L., Nyberg, L., Winblad, B., Nordberg, A., & Almkvist, O. (1999). Brain regions associated with episodic retrieval in normal aging and Alzheimer's disease. Neurology, 52, 1861-1870.

De Groot, J. C., de Leeuw, F.-E., Oudkerk, M., van Gijn, J., Hofman, A., Jolles, J., & Breteler, M. M. B. (2000). Cerebral white matter lesions and cognitive function: The Rotterdam Scan Study. Annals of Neurolology, 47, 145-151.

De la Torre, J. C. (1999). Critical threshold cerebral hypoperfusion causes Alzheimer's disease? Acta Neuropathologica, 98, 1-8.

DeLuca, J., Chelune G. J., Tulski D. S., Lengenfelder, J., & Chiaravalloti N. D. (2004). Is speed processing or working memory the primary information processing deficit in multiple sclerosis? Journal of Clinical and Experimental Neuropsychology, 26, 550-62.

Denckla, M. B., & Rudel, R. (1974). Rapid “automatized” naming of pictured objects, color, letters, and numbers by normal children. Cortex, 1, 186-202.

Fabrigoule, C., Rouch, I., Taberly, A., Letenneur, L., Commenges, D., Mazaux, J.-M., Orgogozo, J.-M., & Dartigues, J.-F. (1998). Cognitive process in preclinical phase of dementia. Brain, 121, 135-141.

Folstein, M., Folstein, S., & McHugh, P. (1975). “Mini Mental State”. A practical method for grading the cognitive state of patients for the clinicians. Journal of Psychiatric Research, 12, 189-198.

Georgiou, G. K., Parrila, R., & Kirby, J. (2006). Rapid naming speed components and early reading acquisition. Scientific Studies of Reading, 10, 199-220.

Girouard, H., & Iadecola, C. (2006). Neurovascular coupling in the normal brain and in hypertension, stroke, and Alzheimer disease. Journal of Applied Physiology, 100, 328-335.

Grossman, M., Zurif, E., Lee, C., Prather, P., Kalmanson, J., Stern, M. B., & Hurtig, H. I. (2002). Information processing speed and sentence comprehenison in Parkinson's disease. Neuropsychology, 16 (2), 174-181.

Hock, C., Villringer, K., Muller-Spahn, F., Wenzel, R., Heekeren, H., Schuh-Hofer, S., Hofmann, M., Minoshima, S., Schwaiger, M., Dirnagl, U., & Villringer, A. (1997). Decrease in parietal cerebral hemoglobin oxygenation during performance of a verbal fluency task in patients with Alzheimer's disease monitored by means of near-infrared spectroscopy (NIRS)—correlation with simultaneously rCBF-PET measurements. Brain Research, 755, 293-303.

Hulme, C., Newton, P., Cowan, N., Stuart, G., & Brown, G. (1999). Think before you speek: pauses, memory search, and trace redintegration processes in verbal memory span. Journal of Experimental Psychology: Learning, Memory, and Cognition, 25, 447-463.

Iadecola, C. (2004). Neurovascular regulation in the normal brain and in Alzheimer's disease. Nature Reviews Neuroscience, 5, 347-360.

Janciauskiene, S M., Erikson, C., Warkentin, S. (2008). A link between sICAM-1, ACE and parietal blood flow in the aging brain. Neurobiology of Aging, (in press).

Junque, C., Pujol, J., Vendrell, P., Bruna, O., Jodar, M., Ribas, J. C., Vinas, J., Capdevila, A., & Marti-Vilalta, J. L. (1990). Leuko-araiosis on magnetic resonance imaging and speed of mental processing. Archives of Neurology, 47, 151-6.

Kircher, T., Brammer, M. J., Levelt, W., Bartels, M., & McGuire, P. K. (2004). Pausing for thought: engagement of left temporal cortex during pauses in speech. NeuroImage, 21, 84-90.

Launer, L. J. Demonstrating the case that AD is a vascular disease: epidemiologic evidence. (2002). Ageing Research Reviews, 1, 61-77.

Maximilian, V. A., Prohovnik, I., & Risberg, J. (1980). The cerebral hemodynamic response to mental activation in normo- and hypercapnia. Stroke, 65-82.

McKhann, G., Drachman, D., Folstein, M., Katzman, R., Price, D., & Stadlan, E. M. (1984). Clinical diagnosis of Alzheimer's disease: Report of the NINCDS-ADRDA Work Group under the auspicies of Department of Health and Human Services Task Force on Alzheimer's Disease. Neurology, 34, 939-944.

Mentis, M. J., Horwitz, B., Grady, C. L., Alexander, G. E., VanMeter, J. W., Maisog, J. M., Pietrini, P., Schapiro, M. B., & Rapoport, S. I. (1996). Visual cortical dysfunction in Alzheimer's desease evaluated with a temporally graded “stress test” during PET. American Journal of Psychiatry, 153, 32-40.

Nebes, R. D. & Madden, D. J. (1988). Different patterns of cognitive slowing produced by Alzheimer's disease and normal aging. Psychological Aging, 3(1), 102-104.

Nebes, R. D., Halligan, E. M., Rosen, J., & Reynolds III, C. F. (1998). Cognitive and motor slowing in Alzheimer's disease and geriatric depression. Journal of the International Neuropsychological Society, 4, 426-434.

Neuhaus, G., Foorman, B. R., Francis, D. J., & Carlson, C. D. (2001 a). The reliability and validity of rapid automatized naming scoring software ratings for the determination of pause and articulation component durations. Educational and Psychological Measurements, 61, 490-504.

Neuhaus, G. F., Foorman, B. R., Francis, D. J., & Carlsson, C. (2001 b). Measures of information processing in Rapid Automatized Naming (RAN) and their relation to reading. Journal of Experimental Child Psychology, 78, 359-373.

Obrist, W. D., Thompson, H. K., Wang, H. S., & Wilkinson, W. E. (1975). Regional cerebral blood flow estimated by 133-Xe inhalation. Stroke, 6, 245-256.

Oldfield, R. C. (1971). The assessment and analysis of handedness. The Edingburgh Handedness Inventory. Neuropsychologia, 9, 97-113.

Perry, R. J., & Hodges, J. R. (1999). Attention and executive deficits in Alzheimer's disease: a critical review. Brain, 122, 383-404.

Peters, N., Opherk, C., Danek, A., Ballard, C., Herzog, J., & Dichgans, M. (2005). The pattern of cognitive performance in CADASIL: a monogenic condition leading to subcortical ischemic vascular dementia. American Journal of Psychiatr, 162(11), 2078-85.

Prins, N. D., van Dijk, E. J., den Heijer, T., Vermeer, S. E., Jolles, J., Koudstaal, P. J., Hofman, A., & Breteler, M. M. (2005). Cerebral small-vessel disease and decline in information processing speed, executive function and memory. Brain, 128, 2034-41.

Risberg, J. (1980). Regional cerebral blood flow measurements by 133-Xe inhalation: Methodology and applications in neuropsychology and psychiatry. Brain and Language, 9, 9-34.

Risberg, J., Ali, Z., Wilson, E. M., Wills, E. L., & Halsey, J. H. (1975). Regional cerebral blood flow by 133-Xe inhalation: Preliminary evaluation of an initial slope index in patients with unstable flow compartments. Stroke, 6, 142-148.

Risberg, J., & Gustafson, L. (1997). Regional cerebral blood flow measurements in the clinical evaluation of demented patients. Dementia and Geriatric Cognitive Disorders, 8, 92-97.

Salthouse, T. A. (1985). Speed of behavior and its implications for cognition. In J. E. Birren & K. Schaie (Eds.), Handbook of the psychology of aging (2nd ed.), pp. 400-426. New York: Van Nostrand Reinhold.

Salthouse, T. A. (1996). The processing theory of adult age differences in cognition. Psychological Review, 103, 403-428.

Salthouse, T. A. (2000). Aging and measures of processing speed. Biological Psychology, 54, 35-54.

Trollor, J. N., Sachdev, P. S., Haindl, W., Brodaty, H., Wen, W., & Walker, B. M. (2006). A high-resolution single photon emission computed tomographic study of verbal recognition memory in Alzheimer's disease. Dementia and Geriatric Cognitive Disorders, 21, 267-274.

Verhaegen, P., & Salthouse T A. (1997). Meta-analyses of age-cognition relations in adulthood: estimates of linear and nonlinear age effects and structural models. Psychological Bulletin, 122, 231-249.

Wakefield, A J., More, L J, Difford, J, McLaughlin, J E. (1994). Immunohistochemical study of vascular injury in acute multiple sclerosis. Journal of Clinical Pathology, 47, 129-133.

Warkentin, S., Risberg, J., Nilsson, A., Karlson, S., & Graae, E. (1991). Cortical activity during speech production. A study of regional cerebral blood flow in normal subjects performing a word fluency task. Neuropsychiatry, Neuropsychology and Behavioural Neurology, 4, 305-316.

Warkentin, S., & Passant, U. (1993). Functional activation of the frontal lobes. Regional cerebral blood flow (rCBF) findings in normals and in patients with frontal lobe dementia performing a word fluency test. Dementia and Geriatric Cognitive Disorders, 4, 188-191.

Warkentin, S., & Passant, U. (1997). Functional imaging of the frontal lobes in organic dementia. Regional cerebral blood flow findings in normals, in patients with frontotemporal dementia and in patients with Alzheimer's disease, performing a word fluency test. Dementia and Geriatric Cognitive Disorders, 8, 105-109.

Warkentin, S., Erikson, C., Janciauskiene, S M. (2008). rCBF pathology in Alzheimer's disease is associated with slow processing speed. Neuropsychologia, 46, 1193-1200.

Woodard, J. L., Grafton, S. T., Votaw, J. R., Green, R. C., Dobraski, M. E., & Hoffman, J. M. (1998). Compensatory recruitment of neural rescources during overt rehearsal of words lists in Alzheimer's disease. Neuropsychology, 12, 491-504.

1-22. (canceled)

23. A method of diagnosing a brain disease induced brain dysfunction of a subject, comprising
registering speech of said subject over a period of time;
determining a pause component of said registered speech; and
determining an occurrence and/or stage of said brain disease induced brain dysfunction from said pause component, wherein said pause component is an accumulated pause time of a total time of said speech correlated to said occurrence and/or stage of said brain dysfunction, and
comparing said pause component with a pre-determined normal pause-component for said diagnosis.

24. A method for assessment of a brain status of a subject, wherein said assessment is performed internally in a system, wherein said brain status comprises a brain disease induced brain dysfunction, wherein said method comprises
analyzing speech of said subject and determining a pause component of said speech; and
determining an occurrence and/or stage of said brain disease induced brain dysfunction in said subject based on said pause component, wherein said pause component is an accumulated pause time of a total time of said speech correlated to said occurrence and/or stage of said brain dysfunction.

25. The method according to claim 24, comprising registering said speech and/or recording said speech of said subject over a period of time; and wherein said analyzing said speech comprises analyzing said registered speech and/or said recorded speech for determining said pause component of said speech.

26. The method according to claim 24, wherein said analyzing said speech of said subject is performed irrespective of a language of said speech.

27. The method according to claim 24, comprising applying a compensation factor for a specific language of said speech for said assessment.

28. The method according to claim 24, comprising applying a compensation factor related to an age of said subject.

29. The method according to claim 24, wherein said assessment is a cognitive test based assessment, comprising the subject freely defining parameters of said cognitive test, for producing said speech.

30. The method according to claim 24 comprising providing a basis for medical personal for deciding if a subject has signs of a brain disease induced brain dysfunction or not, based on said pause component.

31. The method according to claim 30, further comprising directing primary health care resources to those subjects who are at high risk for having a brain disease induced brain dysfunction, and who need further assessment for their diagnosis, while saving financial costs for unnecessary evaluations of patients with negative test results.

32. The method according to claim 24, comprising basing said occurrence and/or stage of said brain disease induced brain dysfunction on a threshold value of said pause time component.

33. The method according to claim 32, wherein said threshold value comprises different ranges for said occurrence and/or stage of said brain disease induced brain dysfunction, and said methods comprises determining
a) a pause time in percent of total speech time of less than or equal to approximately 50% of the total time used by the subject to name a predefined set of color and shape combinations for a healthy subject;
b) a pause time, in percent the total time used by the subject to name a predefined set of color and shape combinations, is longer than approximately 50 to 60% for a subject at risk or in an early stage of the brain disease induced brain dysfunction or a subject suffering from a brain disease induced brain dysfunction.

34. The method according to claim 24, wherein said method comprises a cognitive test performed by said subject, wherein pause time component comprises a mean duration of the pause time measured in relation to the total duration of a naming time of said cognitive test performed by said subject.

35. The method according to claim 34, further comprising determining said occurrence and/or stage of said brain disease induced brain dysfunction by comparing said total duration of said total naming time and a total pause duration with normal reference values.

36. The method according to claim 34, comprising determining said occurrence and/or stage of said brain disease induced brain dysfunction from said pause component by calculating at least one index on the relation between total pause duration, pause-articulation time, in percent or in seconds.

37. The method according to claim 34, wherein said determining said occurrence and/or stage of said brain disease induced brain dysfunction from said pause component does not comprise registering of naming errors.

38. The method according to claim 24, wherein said determining said occurrence and/or stage of said brain disease induced brain dysfunction from said pause component comprises associating a slowing of speech compartments with white matter function/dysfunction and/or cerebrovascular dysfunction, in either healthy aging, mild cognitive impairment (MCI) or dementia.

39. The method according to claim 24, wherein said assessment is cognitive test based assessment, wherein the subject is free to define parameters of said cognitive test, wherein said cognitive test provides measures of processing speed, such as for example using simple colors and shapes, or naming other defined stimuli, is non-invasive.

40. The method according to claim 39, wherein said cognitive test is implemented in an education and culture-free manner, and wherein said cognitive test does not comprise questions related to knowledge of said subject.

41. The method according to claim 24, wherein said brain disease induced brain dysfunction is not of developmental origin of the central nervous system (CNS), but reflects the aging and disease processes of the CNS in the elderly.

42. The method according to claim 24, further comprising determining a level of vitamin B12 in said subject via determination of said pause component.

43. The method according to claim 24, further comprising determining a level of folate in said subject via determination of said pause component.

44. The method according to claim 24, wherein said brain disease induced brain dysfunction is related to dementia, such as Alzheimer's disease; Multiple sclerosis (MS); an subcortical white matter disease or demyelinating disease; HIV; malaria; cerebrovascular disease (VaD); encephalitis; traumatic brain injury (TBI); or mild cognitive impairment (MCI); traumatic brain injury (TBI); effects of street drugs; alcohol abuse; or side effects of prescribed drugs; pharmaceutical drug treatments, such as CNS, heart, lung or otherwise.

45-53. (canceled)

54. The method according to claim 24 comprising assessing any training effects on pause time duration, performed by a subject, either by physical training and exercise to improve brain blood flow and brain oxygenation and/or by any mental training programs which are aimed to improve any cognitive abilities, such as for example memory function and reading and writing abilities, of that subject.

55. The method according to claim 24 comprising assessing the effects on pause time duration of any nutritional supplementations used by the subject, with supplementation is aimed to improve the physical and/or mental well-being of that subject. Such supplementations may involve any vitamin supplementation and any supplementation of any polyunsaturated fatty acids aimed to improve the lipid metabolism of the brain of that subject.

56. The method according to claim 24 comprising assessing the effects on pause time duration of any pharmaceutical intervention approach aimed at improving the transmission of any neurotransmitter subservient to any mental processes performed by the brain, such as for example any pharmaceutical drug for the treatment of dementia disorders.

57. The method according to claim 24 comprising assessing the effect on pause time duration by reducing the build-up of toxic by-products within the brain and/or to increase the elimination of toxic waste products of metabolism in the brain, via the blood-brain barrier and/or via the blood-cerebrospinal fluid barriers of the brain.

58. The method according to claim 24 comprising assessing the effects on pause time duration of any pharmaceutical and/or genetic intervention approach aimed at influencing or manipulating the cleavage processes by protease inhibitors of the amyloid precursor protein (APP).

59. The method according to claim 24 comprising assessing effects on pause time duration of pharmaceutical or genetic approach aimed to improve the symptoms of Parkinson's disease and Parkinson's dementia which affect any neurotransmitter system in the brain which overlaps with neurotransmitter systems that degenerate in Alzheimer's disease, dementia with Lewy bodies, and Frontotemporal dementia.

60. The method according to claim 24 comprising assessing the effects on pause time duration of disorders that slow down the brain's ability to process information, such as tumor or stoke.

61. The method according to claim 24 comprising assessing the effect on pause time duration of metabolic or other dysfunction in other bodily organs than the brain of the subject, which affect the cognitive performance of the brain.
